(19) United States
(12) Patent Application Publication
(10) Pub. No.: US 2013/0281868 A1
(43) Pub. Date: Oct. 24, 2013

(54) BLOOD PRESSURE MEASUREMENT DEVICE

(71) Applicant: DENSO CORPORATION, Kariya-city (JP)

(72) Inventors: Taiji KAWACHI, Kariya-city (JP); Mitsuo OKUMURA, Aichi-gun (JP); Kyo YAMAMOTO, Nagoya-city (JP); Tokio HARUTA, Nagoya-city (JP)

(73) Assignee: DENSO CORPORATION, Kariya-city (JP)

(21) Appl. No.: 13/866,154

(22) Filed: Apr. 19, 2013

(30) Foreign Application Priority Data
Apr. 24, 2012 (JP) 2012-98749

(51) Int. Cl. A61B 5/021

(52) U.S. Cl. CPC A61B 5/02141
USPC 600/485

(57) ABSTRACT

A blood pressure measurement device includes a case, an electrocardiogram electrode, a pulse wave sensor, an estimation portion, and a display portion. The case has a peripheral surface to be held with both hands. The electrocardiogram electrode detects an electrocardiogram signal associated with a movement of a heart through at least one of the hands. The pulse wave sensor detects a pulse wave signal associated with the movement of the heart through a least one of the hands. The estimation portion estimates a blood pressure based on the electrocardiogram signal and the pulse wave signal. The display portion displays the blood pressure estimated by the estimation portion.

### CROSS REFERENCE TO RELATED APPLICATIONS

The present application is based on and claims priority to Japanese Patent Application No. 2012-98749 filed on Apr. 24, 2012, the contents of which are incorporated in their entirety herein by reference.

### TECHNICAL FIELD

The present disclosure relates to a blood pressure measurement device.

### BACKGROUND

As an example of a blood pressure measurement device, JP 2007-75586 A (corresponding to US 2007/0173726 A1) discloses a bio-signal measurement device that includes a horizontal case as a main body. The horizontal case has palm support portions on which both hands are respectively put. The palm support portions are separated from each other by a shoulder width of an average adult. Each of the palm support portions includes a first electrode (third electrode) that comes into contact with a ball of a thumb, and a second electrode (fourth electrode) that comes into contact with a hypothenar. One of the palm support portions includes a blood pressure cuff. The blood pressure cuff corresponds to a part of a finger and detects a blood pressure. Using the bio-signal measurement device, biological information including a blood pressure and a heart rate can be simply detected only by putting both open palms on the palm support portions and inserting a part of a finger into the blood pressure cuff. The detected biological information can be confirmed through a display in a vertical case that is protruded from a middle of the horizontal case.

However, the bio-signal measurement device has a typical appearance of a measurement device and is not suitable for usage in homes. Thus, it is desirable to develop a blood pressure meter that looks natural in homes and has a shape adapted to an interior decoration. Also in a medical institution, such as a hospital, it is desirable to develop a blood pressure meter with which a subject can simply measure a blood pressure by oneself without help from a doctor or a nurse.

### SUMMARY

It is an object of the present disclosure to provide a blood pressure measurement device with which a subject can simply measure a blood pressure in home or a hospital.

A blood pressure measurement device according to an aspect of the present disclosure includes a case, an electrocardiogram electrode, a pulse wave sensor, an estimation portion, and a display portion. The case has a peripheral surface to be held with both hands. The electrocardiogram electrode detects an electrocardiogram signal associated with a movement of a heart through at least one of the hands that hold the case. The pulse wave sensor detects a pulse wave signal associated with the movement of the heart through a least one of the hands that hold the case. The estimation portion estimates a blood pressure based on the electrocardiogram signal detected by the electrocardiogram electrode and the pulse wave signal detected by the pulse wave sensor. The display portion displays the blood pressure estimated by the estimation portion.

A subject can measure a blood pressure only by holding the blood pressure with both hands. Thus, a subject can simply measure the blood pressure in home or a hospital.

### BRIEF DESCRIPTION OF THE DRAWINGS

Additional objects and advantages of the present disclosure will be more readily apparent from the following detailed description when taken together with the accompanying drawings. In the drawings:

FIG. 1 a perspective view of a blood pressure measurement device according to a first embodiment of the present disclosure;

FIG. 2A is a front view of the blood pressure measurement device, FIG. 2B is a plan view of the blood pressure measurement device, FIG. 2C is a side view of the blood pressure measurement device, FIG. 2D is a back view of the blood pressure measurement device, and FIG. 2E is a bottom view of the blood pressure measurement device;

FIG. 3A is a front view showing a state where the blood pressure measurement device is held with both hands, FIG. 3B is a plane view showing the state where the blood pressure measurement device is held with the both hands;

FIG. 4A is a diagram showing specs of the blood pressure measurement device, and FIG. 4B is a perspective view of a blood pressure measurement device that does not satisfy a spec of a thickness;

FIG. 5A is a cross-sectional view of an electrocardiogram electrode, FIG. 5B is a diagram for explaining an S/N ratio of an electrocardiogram, and FIG. 5C is a graph showing a relationship of a thickness of the electrocardiogram electrode and a ratio of the S/N ratio being greater than or equal to 2;

FIG. 6 is a diagram showing display contents of a display portion;

FIG. 7 is a diagram showing a calculation program executed by a control portion;

FIG. 8 is a graph showing an electrocardiogram signal and a pulse wave signal;

FIG. 9 is a graph showing a pulse wave signal, a signal of a first order differential, and a signal of a second order differential;

FIG. 10 is a side view of a blood pressure measurement device according to a second embodiment of the present disclosure;

FIG. 11A is a perspective view showing a state where a blood pressure measurement device according to a third embodiment of the present disclosure is held with both hands, FIG. 11B is a front view of the blood pressure measurement device, FIG. 11C is a side view of the blood pressure measurement device, and FIG. 11D is back view of the blood pressure measurement device;

FIG. 12A is a perspective view showing a state where a blood pressure measurement device according to a fourth embodiment of the present disclosure is held with both hands, and FIG. 12B is a side view of the blood pressure measurement device;

FIG. 13 is a side view of a blood pressure measurement device according to a fifth embodiment of the present disclosure; and

FIG. 14 is a back view of a blood pressure measurement device according to a sixth embodiment of the present disclosure.

### DETAILED DESCRIPTION

Embodiments of the present disclosure will be described with reference to the accompanying drawings.

### First Embodiment

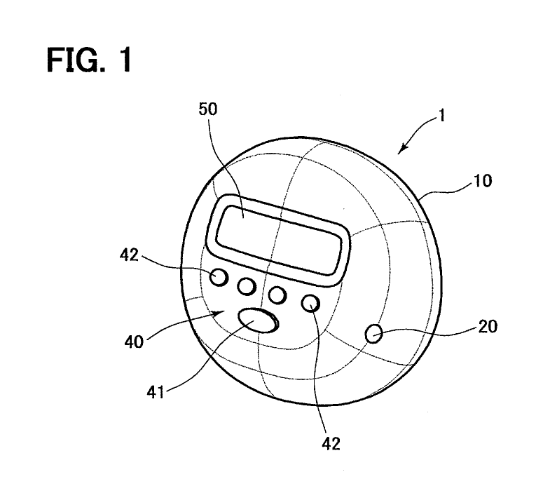

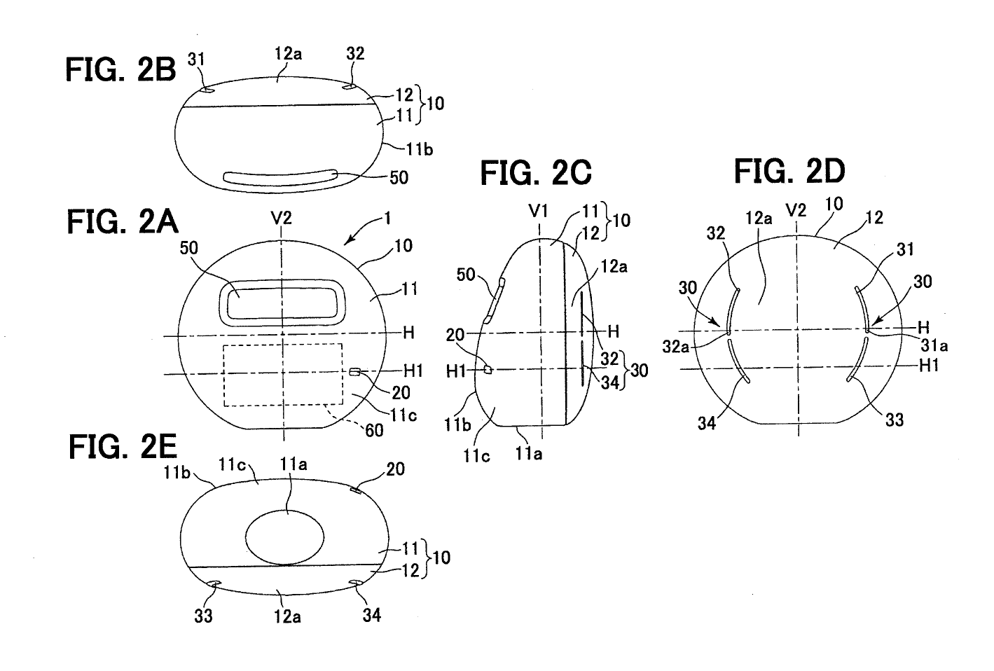

A blood pressure measurement device 1 according to a first embodiment of the present disclosure detects an electrocardiogram signal and a pulse wave signal of a subject, calculates a blood pressure and the like based on the electrocardiogram signal and the pulse wave signal, and displays the calculated blood pressure and the like. As shown in FIG. 1 to FIG. 3B, the blood pressure measurement device 1 includes a case 10, a pulse wave sensor 20, an electrocardiogram electrode 30, an operation switch 40, a display portion 50, and a control portion 60. FIG. 2A and subsequent drawings, the operation switch 40 is not illustrated.

The case 10 has a spherical shape. In the present application, the “spherical shape” is not limited to a spherical shape in geometry in the strict sense, and may include a shape in which a transverse section or a longitudinal section has an egg shape, an elliptical shape, a protruding closed smooth curve similar to an elliptical shape, and an oval shape. For example, a front shape of the case 10 is close to a circular shape (see FIG. 2A), a planar shape of the case 10 is close to an elliptical shape (see FIG. 2B), and a side shape of the case 10 is close to an egg shape (see FIG. 2C). The case 10 includes a case body 11 and a lid 12. The case body 11 has a flat bottom portion 11a. The case 10 stands up by placing the bottom portion 11a of the case body 11 on a table.

The case body 11 and the lid 12 houses a circuit substrate including the control portion 60 (see a dashed line in FIG. 2A). The case body 11 is disposed on the front side, and the lid 12 is disposed on the backside (see FIG. 2B, FIG. 2C). When a horizontal plane that bisects the case 10 in a height direction of the case 10 is expressed as a horizontal plane H, and a vertical plane that bisects a thickness of the case 10 on the horizontal plane H in the front-back direction is expressed as a vertical plane V1, the lid 12 is disposed behind the vertical plane V1. In addition, when a vertical plane that bisects a thickness of the case 10 on the horizontal plane H in a right-left direction is expressed as a vertical plane V2, the case 10 is line symmetric with respect to the vertical plane V2.

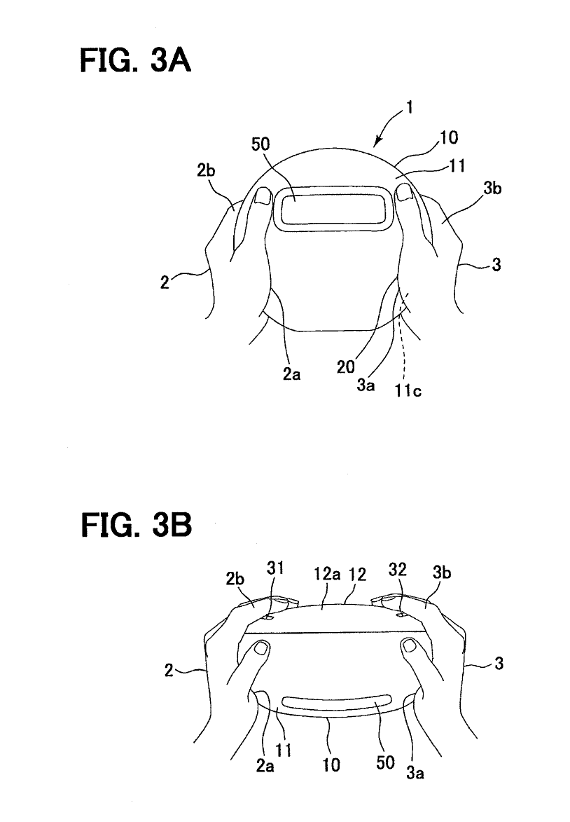

As shown in FIG. 2C, the case body 11 includes a top portion 11b and a peripheral surface 11c. The top portion 11b protrudes foremost at a position below the horizontal plane H. The peripheral surface 11c is located below the top portion 11b. The peripheral surface 11c has a curve shape similar to a natural curve of a relaxing palm of the subject and a normal line of the curve shape extends downward. Thus, as shown in FIG. 3A and FIG. 3B, when the subject holds the case 10 with both hands 2, 3, it is a natural style that the subject holds the case body 11 from an obliquely lower position by bringing palms 2a, 3a into contact with the peripheral surface 11c of the case body 11.

In other words, when the subject holds the case 10 with the both hands 2, 3, according to a natural bending action of the palms 2a, 3a and fingers 2b, 3b, the palms 2a, 3a and base end portions of the fingers 2b, 3b come into plane contact with the peripheral surface 11c of the case body 11, and leading end portions and middle portions of the fingers 2b, 3b come into plane contact with a peripheral surface 12a of the lid 12. Because the subject does not grip the case 10, the subject does not put excessive power into the both hands 2, 3, and the subject can stably measure the blood pressure and the like.

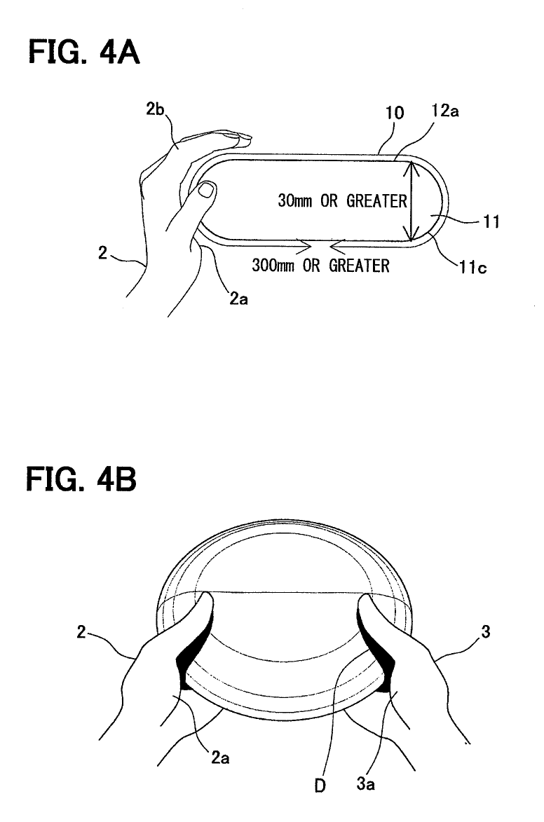

As shown in FIG. 4A, the case 10 has such a shape that a thickness of the peripheral surface 11c in the front-rear direction is greater than or equal to 30 mm, and a circumference of the peripheral surface 11c is greater than or equal to 300 mm. In a case where the thickness of the peripheral surface 11c in the front-rear direction is less than 30 mm, as shown in FIG. 4B, a gap D may be left between the case body 11 and the palms 2a, 3a, and it becomes difficult to secure a stable contact of the pulse wave sensor 20 and the palm 3a. However, in a case where the thickness is greater than or equal to 30 mm, even for a man with an average hand length, a gap D is less likely to be left between the case body 11 and the palms 2a, 3a. The hand length is a length from a leading end of a middle finger to a wrist. The average hand length of 60-64 years old men is about 191 mm and the average hand length of 60-64 years old women is about 178 mm. The thickness of the peripheral surface 11c in the front-rear direction may be 250 mm at the maximum.

In a case where the circumference of the peripheral surface 11c is less than 300 mm, when the case 10 is held with the both hands 2, 3, the fingers 2b, 3b may come into contact with each other, and it becomes difficult to detect a normal electrocardiogram signal with the electrocardiogram electrode 30. However, in a case where the circumference is greater than or equal to 300 mm, even for the man with the average hand length, the fingers 2b, 3b are less likely to come into contact with each other when the case 10 is held with the both hands 2, 3. The circumference of the peripheral surface 11c may be 1000 mm at the maximum.

The pulse wave sensor 20 is a known optical reflection sensor that includes a light emitting element (e.g., a light emitting diode) and a light receiving element (e.g., a photo diode). When the light emitting element emits a light toward a hand of the subject, a part of the light is absorbed by hemoglobin in blood that flows in an arteriole in a body of the subject, the remaining light is reflected and scattered at the arteriole, and a part of the scattered light enters the light receiving element. The amount of hemoglobin that flows in the arteriole changes in a wavy manner due to pulsation of the blood, and the amount of light absorbed by the hemoglobin also changes in a wavy manner. Accordingly, the amount of light reflected at the arteriole and detected by the light receiving element changes, and the change of the amount of light detected by the light receiving element is transmitted to the control portion 60 as the pulse wave signal (e.g., a voltage signal).

The pulse wave sensor 20 is disposed at a position corresponding to one of the palms 2a, 3a that hold the case 10. For example, the pulse wave sensor 20 is disposed at a portion of the case body 11 below the horizontal surface H and in front of the vertical plane V1. As shown in FIG. 2C, the pulse wave sensor 20 is disposed at an upper position of the top portion 11b that protrudes foremost in the case body 11, and the pulse wave sensor 20 is embedded in a position corresponding to a ball of a thumb in the palm 3a at a time when the case 10 is held with the both hands 2, 3. The position of the pulse wave sensor 20 is not limited to the position corresponding to the ball of the thumb in the palm 3a and may also be a position corresponding to a ball of a thumb in the palm 2a.

When the pulse wave signal is detected with the pulse wave sensor 20, the pulse wave sensor 20 needs to be pressed at an appropriate force. The pulse wave sensor 20 needs to be applied with a force to bring a skin of the palm 3a (2a) into contact with the pulse wave sensor 20 with certainty. However, when an excessive force is applied, a blood vessel under the skin may be crushed, and the pulse wave signal cannot be detected. In a case where the pulse wave sensor 20 is pressed with the thumb, a force applied to the pulse wave sensor 20 can be easily adjusted. However, because the subject needs to put the thumb on the pulse wave sensor 20 and apply a constant and appropriate force to the pulse wave sensor 20, the thumb may tremble and noise may be generated.

In a case where the pulse wave sensor 20 is disposed at the position corresponding to the ball of the thumb in the palm 3a (2a), the subject can bring the palm 3a (2a) into contact with the pulse wave sensor 20 only by holding the case 10 with the both hands 2, 3 and needs not be conscious of the existence of the pulse wave sensor 20. Thus, the subject can continuously apply a constant and appropriate force to the pulse wave sensor 20.

The electrocardiogram electrode 30 detects an electrocardiogram signal (a signal based on a potential difference between electrodes) and transmits the electrocardiogram signal to the control portion 60. As shown in FIG. 2C and FIG. 2D, the electrocardiogram electrode 30 includes a left hand electrode 31, a right hand electrode 32, and a pair of intermediate electrodes 33, 34. The left hand electrode 31 and the right hand electrode 32 are disposed at positions corresponding to the fingers 2b, 3b of the both hands 2, 3 that hold the case 10. Each of the electrodes 30-34 is disposed on the lid 12.

When a plane passing through the pulse wave sensor 20 and being parallel with the horizontal plane H is expressed as a horizontal plane H1, at least a part of each of the left hand electrode 31 and the right hand electrode 32 is disposed above the horizontal plane H1. Accordingly, when the subject holds the case 10 with the both hands 2, 3 and the palms 2a, 3a are in close contact with the peripheral surface 11c of the case 10 from the obliquely lower portion, the finger 2b can certainly come into contact with the left hand electrode 31, and the finger 3b can certainly come into contact with the right hand electrode 32 even if a holding position of the palms 2a, 3a changes.

The intermediate electrodes 33, 34 are disposed below the horizontal plane H. Accordingly, when the subject holds the case 10 with the both hands 2, 3 and holds the case body 11 from the obliquely lower position by bring the palms 2a, 3a into close contact with peripheral surface 11c of the case body 11, the finger 2b can certainly come into contact with the left hand electrode 31 and the intermediate electrode 33, and the finger 3b can certainly come into contact with the right hand electrode 32 and the intermediate electrode 34. The intermediate electrodes 33, 34 establish a short circuit in the case 10 and operate as one electrode. Signals detected at the electrodes 31-34 are amplified, for example, by an operational amplifier. Accordingly, noise due to a body motion can be effectively removed.

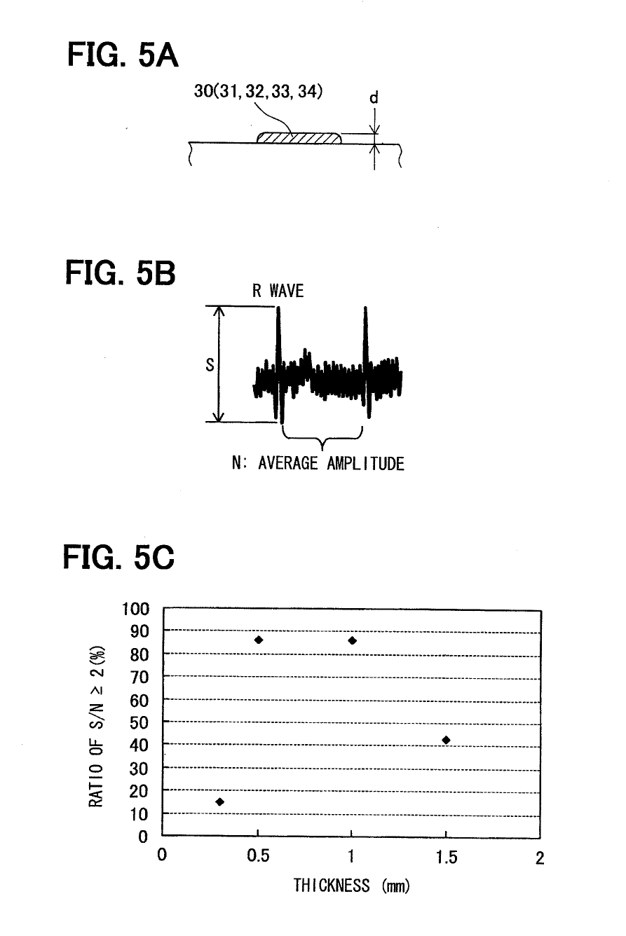

As shown in FIG. 2D, each of the electrodes 31-34 has an arc shape, and a depressed portion of each arc faces to a center portion of the lid 12. As shown in FIG. 5A, each of the electrodes 31-34 is embedded in the case 10 in such a manner that a protruding height d is within a range from 0.5 mm to 1.0 mm.

As shown in FIG. 5B, when an amplitude of an R-wave in an electrocardiogram is expressed as S and an average amplitude of a part in the electrocardiogram other than the R-wave is expressed as N, extraction of the R-wave becomes easy with increase in S/N ratio. Electrodes having thicknesses of 0.3 mm, 0.5 mm, 1.0 mm, 1.5 mm are prepared, the electrocardiogram signal for 10 beats are measured for each of the electrodes, and a ratio of the S/N ratio being greater than or equal to 2 is calculated. As shown in FIG. 5C, the best result can be obtained in cases where the thickness is 0.5 mm and 1.0 mm. In other words, when the protruding height of each of the electrodes 31-34 from the lid 12 is set to be within a range from 0.5 mm to 1.0 mm, the S/N ratio in the electrocardiogram can be large. In order to secure electrical impedance, an area of a protruding plane of each of the electrodes 31-34 is set to be about 1 cm².

The operation switch 40 is, for example, a pressing button. The operation switch 40 includes a measurement start switch 41 for starting measurement and a selection switch 42 for inputting values of personal data by the subject (see FIG. 1). The operation switch 40 is not limited to the pressing button and may also be a touch switch that detects a contact of a finger based on a change in electrostatic capacity.

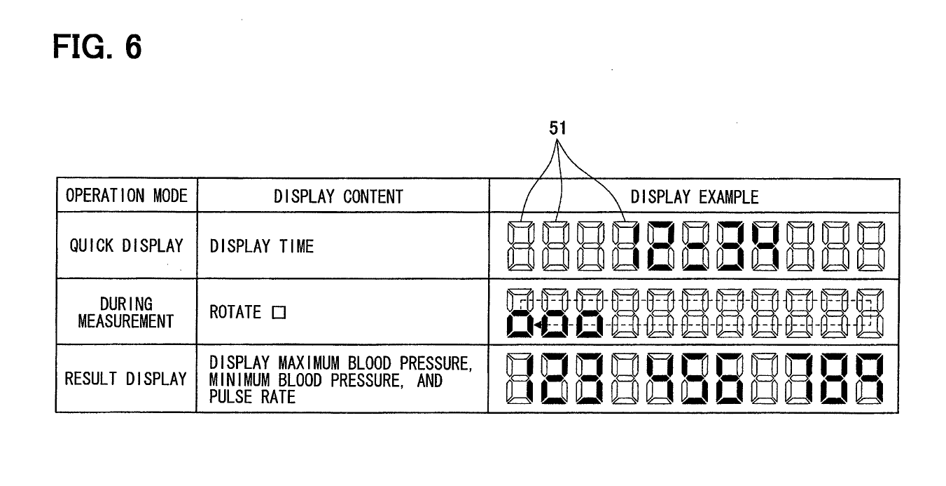

As shown in FIG. 6, the display portion 50 includes, for example, 7-segment LED 51. The display portion 50 is disposed above the horizontal plane H and in front of the vertical plane V1 so that the subject can easily see the display portion 50. The display portion 50 displays the measurement result, that is, the blood pressure (the maximum blood pressure and the minimum blood pressure) and the pulse rate of the subject calculated at the control portion 60. The display portion 50 is not limited to a display that includes the 7-segment LED 51. The display portion 50 may be a liquid crystal display or an organic light emitting display.

The display portion 50 displays a time when the blood pressure measurement device 1 is not used as the blood pressure meter. In addition, the display portion 50 has a display function to inform that it is during measurement until the display portion 50 displays the measurement result. For example, the display portion 50 lights a part of the 7-segment LED 51 so as to form a predetermined mark, and informs that it is during measurement by moving the mark around. If the display portion 50 displays nothing until the measurement result is obtained, the subject cannot discriminate whether it is during measurement or an operation is stopped, and the subject may have a feeling of anxiety. Thus, the display portion 50 displays the predetermined mark during measurement.

The control portion 60 includes a microcomputer, an input interface circuit, and an output interface circuit. The microcomputer includes a central processing unit (CPU), a read only memory (ROM), and a random access memory (RAM). The control portion 60 executes a calculation program stored in the ROM. The control portion 60 calculates the blood pressure and the pulse rate based on the electrocardiogram signal from the electrocardiogram electrode 30 and the pulse wave signal from the pulse wave sensor 20, which are acquired through the input interface circuit. The control portion 60 stores calculated values in the RAM and displays the calculated value in the display portion 50. The control portion 60 can operate as an estimation portion.

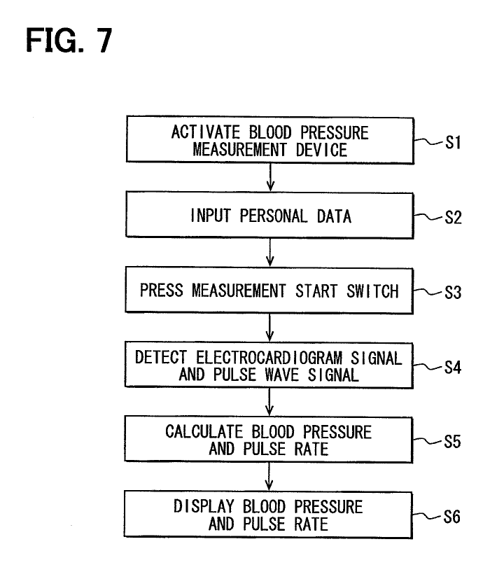

Next, an operation of the blood pressure measurement device 1 will be described. When the measurement start switch 41 in the operation switch 40 is turned on, the control portion 60 starts to execute the calculation program shown in FIG. 7.

The blood pressure measurement device 1 is activated when the power source is turned on (S1). The subject inputs personal data using the selection switch 42 as necessary (S2). After the measurement start switch 41 is turned on (S3), the subject holds the case 10 with the both hands 2, 3. Then, the electrocardiogram electrode 30 detects the electrocardiogram signal through the palm 3a, and the pulse wave sensor 20 detects the pulse wave signal through the fingers 2b, 3b (S4).

The control portion 60 calculates the blood pressure and the pulse rate based on the electrocardiogram signal from the electrocardiogram electrode 30 and the pulse wave signal from the pulse wave sensor 20. A method of calculating the blood pressure and the pulse rate based on the electrocardiogram signal and the pulse wave signal is disclosed, for example, in JP-A-2009-089829. In the present embodiment, the blood pressure and the pulse rate are calculated using the method.

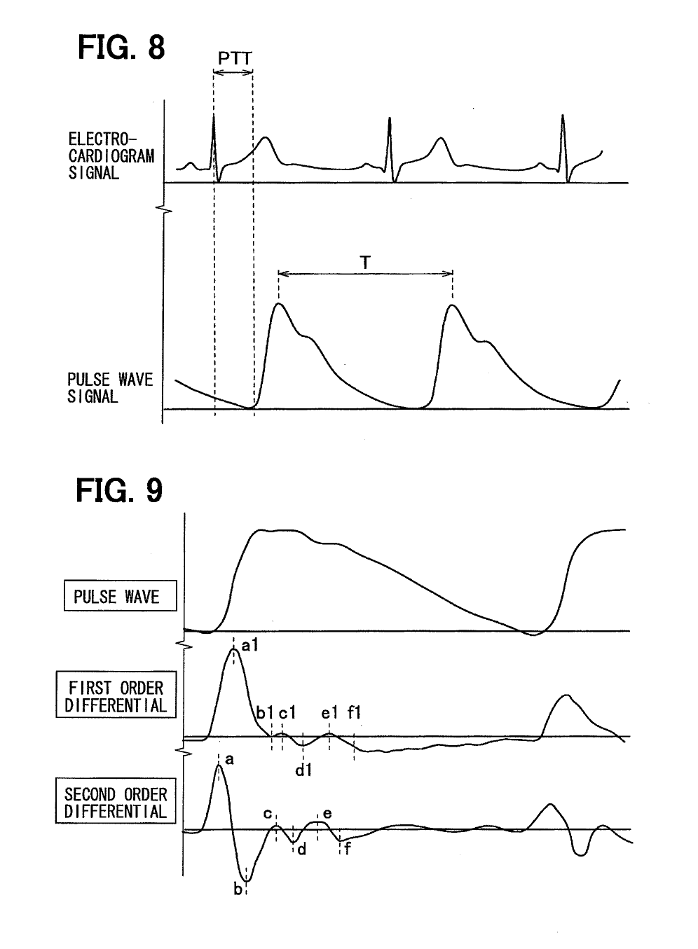

Specifically, as shown in FIG. 8, the control portion 60 compares the electrocardiogram signal and the pulse wave signal and calculates a pulse wave propagation time PTT that is a delay time of the pulse wave signal with respect to the electrocardiogram signal. The control portion 60 calculates a pulse wave period T by analyzing the pulse wave signal. As shown in FIG. 9, the control portion 60 performs a first order differential (velocity pulse wave) and a second order differential (acceleration pulse wave) and calculates the maximum value and the minimum value of feature quantities of each differential. In the velocity pulse wave, the feature quantities are points a1-f1 in FIG. 9. In the acceleration pulse wave, the feature quantities are points a-f in FIG. 9.

Next, the control portion 60 analyzes the pulse wave signal and classifies into a young type or an old type. When a value of determination expression (b-c-d-e)/a using the feature quantities a-e of the acceleration pulse wave is less than or equal to a predetermined determination value (e.g., −0.5), the control portion 60 determines that the pulse wave is the young type and calculates the blood pressure BP using the following equation (1). When the value of determination expression (b-c-d-e)/a is greater than the predetermined determination value, the control portion determines that the pulse wave is the old type and calculates the blood pressure BP using the following equation (2). In each of the equations (1), (2), a weight W included in the personal data can be omitted.

BP=α_{y}·PTT+β_{y}·d+γ_{y}·W+ (1)

BP=αtₒ·PTT+βₒ·d+γₒ·W+ (2)

where each of α_{y}, β_{y}, γ_{y}, αₒ, βₒ, γₒ indicates a factor, and the W indicates the weight.

In addition, the control portion 60 calculates the pulse rate PR from the calculated pulse period T (second) based on the following equation (3).

PR=60/T (3)

After the control portion 60 executes a process at S5 in FIG. 7, the control portion 60 instructs the display portion 50 to display the calculated blood pressure BP and the calculated pulse rate PR through the output interface circuit (S6). In the present case, the display portion 50 displays the maximum blood pressure and the minimum blood pressure as the blood pressure BP in a manner similar to a conventional blood pressure meter.

As is clear from the above description, the subject can measure the blood pressure only by holding the blood pressure measurement device 1 with the both hands 2, 3. Thus, the blood pressure measurement device 1 can make measurement of the blood pressure simple and can be suitably used in home or a hospital. Furthermore, because the case 10 has a spherical shape, the blood pressure measurement device 1 can be adapted to an interior design, and is appropriate for usage in home.

### Second Embodiment

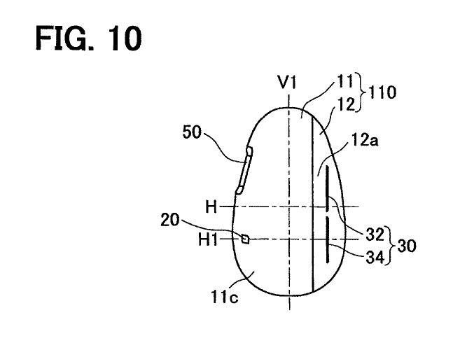

A blood pressure measurement device 1 according to a second embodiment of the present disclosure will be described with reference to FIG. 10. In the first embodiment, the case 10 is asymmetric with respect to the vertical plane V1. The blood pressure measurement device 1 according to the present embodiment includes a case 110 that is symmetric with respect to the vertical plane V1 and is closer to an egg shape than the case 10. In the blood pressure measurement device 1 according to the present embodiment, because a configuration excluding the shape of the case 110 is similar to the configuration of the blood pressure measurement device 1 according to the first embodiment, corresponding components are denoted by the same reference numerals and description about the corresponding components is omitted.

Also with the blood pressure measurement device 1 according to the present embodiment, the blood pressure and the like can be simply detected.

### Third Embodiment

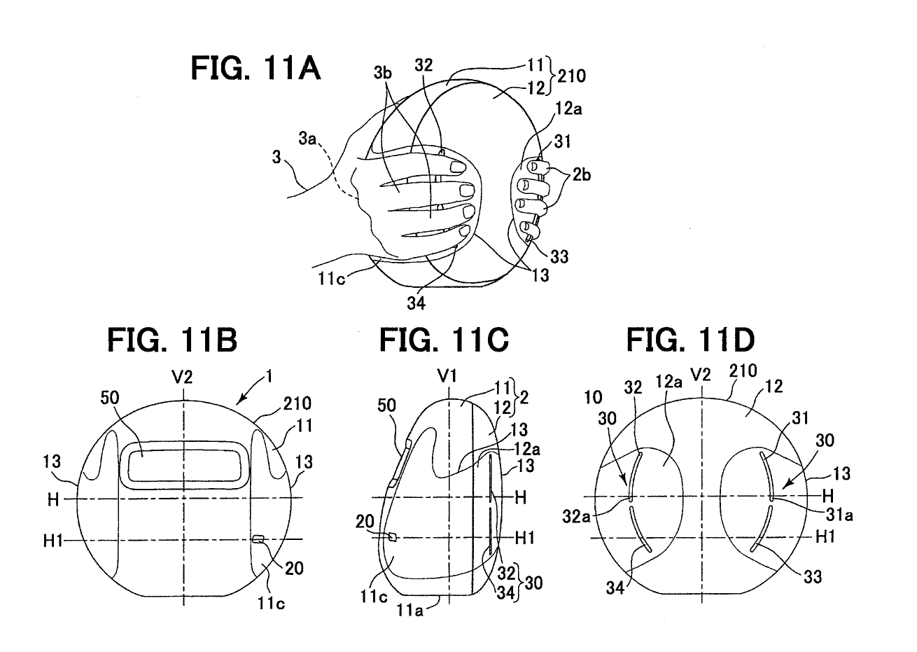

A blood pressure measurement device 1 according to a third embodiment of the present disclosure will be described with reference to FIG. 11A to FIG. 11D. The blood pressure measurement device 1 according to the present embodiment includes a case 210. The case 210 includes a guide portion 13 that informs the subject of holding positions of the palms 2a, 3a and/or the fingers 2b, 3b. The guide portion 13 may be formed by forming a uneven surface (difference in level) in the case 210 at positions corresponding to the holding positions of the palms 2a, 3a and/or the fingers 2b, 3b or the guide portion 13 may be formed by printing or attaching a seal member at the positions corresponding to the holding positions. In the blood pressure measurement device 1 according to the present embodiment, because a configuration excluding the guide portion 13 is similar to the configuration of the blood pressure measurement device 1 according to the first embodiment, corresponding components are denoted by the same reference numerals and description about the corresponding components is omitted.

When the case 210 has the uneven surface, the subject can easily notice the positions of the peripheral surface 11c of the case body 11 and the peripheral surface 12a of the lid 12 tactually. When the guide portion 13 is formed by printing or attaching a seal, the subject can easily notice the positions of the peripheral surface 11c, 12a of the lid 12 visually. In either case, a usability of the blood pressure measurement device 1 can be improved.

### Fourth Embodiment

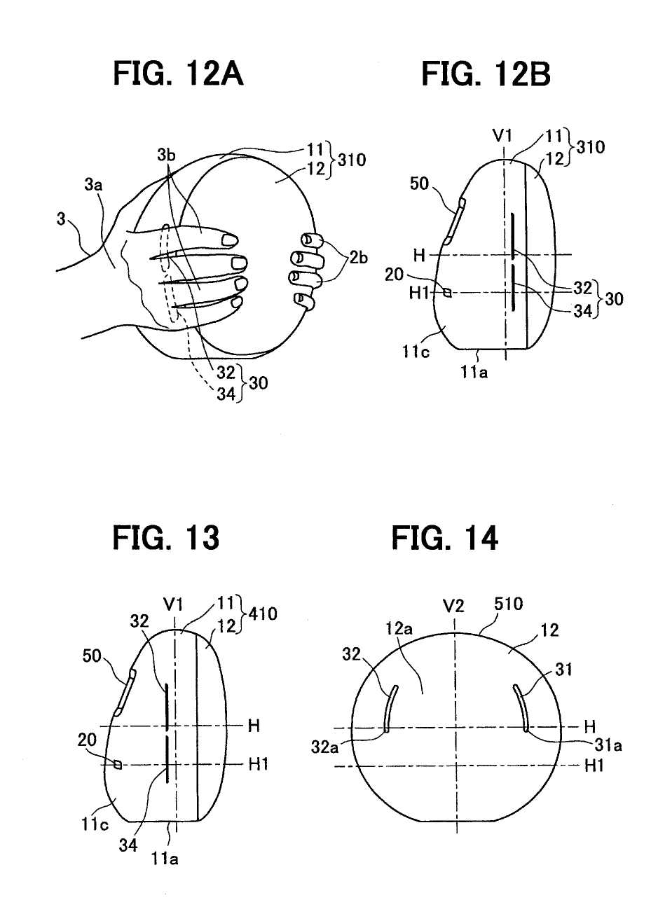

A blood pressure measurement device 1 according to a fourth embodiment of the present disclosure will be described with reference to FIG. 12A and FIG. 12B. The blood pressure measurement device 1 includes a case 310. In the case 310, the electrocardiogram electrode 30 is disposed in the case body 11. In FIG. 12A and FIG. 12B, only the right hand electrode 32 and the intermediate electrode 34 corresponding to the finger 3b of the right hand 3 are illustrated. However, also the left hand electrode 31 and the intermediate electrode 33 corresponding to the finger 2b of the left hand 2 are formed in the case body 11 in a manner similar to the right hand electrode 32 and the intermediate electrode 34. In the blood pressure measurement device 1 according to the present embodiment, because a configuration excluding the electrocardiogram electrode 30 is similar to the configuration of the blood pressure measurement device 1 according to the first embodiment, corresponding components are denoted by the same reference numerals and description about the corresponding components is omitted.

In the present embodiment, the electrocardiogram electrode 30 is disposed behind the vertical plane V1 so as to correspond to the fingers 2b, 3b of the both hands 2, 3. Also with the blood pressure measurement device 1 according to the present embodiment, the blood pressure and the like can be simply measured.

### Fifth Embodiment

A blood pressure measurement device 1 according to a fifth embodiment of the present disclosure will be described with reference to FIG. 13. The blood pressure measurement device 1 according to the present embodiment includes a case 410. In the case 410, the electrocardiogram electrode 30 is disposed in front of the vertical plane V1. In the blood pressure measurement device 1 according to the present embodiment, because a configuration excluding the electrocardiogram electrode 30 is similar to the configuration of the blood pressure measurement device 1 according to the first embodiment, corresponding components are denoted by the same reference numerals and description about the corresponding components is omitted.

In the present embodiment, the electrocardiogram electrode 30 corresponds to the fingers 2b, 3b of the both hands 2, 3 or the palms 2a, 3a. Because contact of the electrocardiogram electrode 30 and the fingers 2b, 3b or the palms 2a, 3a can be secured, the blood pressure and the like can be easily measured with the blood pressure measurement device 1 according to the present embodiment.

In the fourth embodiment and the fifth embodiment, the blood pressure measurement device 1 has a shape similar to the blood pressure measurement device 1 described in the first embodiment. However, also in the blood pressure measurement device 1 according to the second embodiment in which the case 110 is symmetric with respect the vertical plane V2 and the blood pressure measurement device 1 according to the third embodiment having the guide portion 13, the position of the electrocardiogram electrode 30 may be changed in a manner similar to the fourth embodiment of the fifth embodiment.

### Sixth Embodiment

A blood pressure measurement device 1 according to a sixth embodiment of the present disclosure will be described. In the first to fifth embodiment, the electrocardiogram electrode 30 includes the intermediate electrodes 33, 34. The blood pressure measurement device 1 according to the present embodiment includes a case 510 in which the intermediate electrodes 33, 34 are omitted. In the blood pressure measurement device 1 according to the present embodiment, a configuration excluding the electrocardiogram electrode 30 is similar to the configuration of the blood pressure measurement device 1 according to the first embodiment. Thus, corresponding components are denoted by the same reference numerals and description about the corresponding components is omitted.

In the first to sixth embodiments, the blood pressure measurement device 1 is configured to measure the blood pressure and the pulse rate. However, the blood pressure measurement device 1 may be configured to measure a body fat in addition to the blood pressure and the pulse rate.

A position where the case body 11 and the lid 12 are fitted is not limited to a position behind the vertical plane V1 and may be changed optionally.

What is claimed is:

1. A blood pressure measurement device comprising:
a case having a peripheral surface to be held with both hands;
an electrocardiogram electrode detecting an electrocardiogram signal associated with a movement of a heart through at least one of the hands that hold the case;
a pulse wave sensor detecting a pulse wave signal associated with the movement of the heart through a least one of the hands that hold the case;
an estimation portion estimating a blood pressure based on the electrocardiogram signal detected by the electrocardiogram electrode and the pulse wave signal detected by the pulse wave sensor; and
a display portion displaying the blood pressure estimated by the estimation portion.

2. The blood pressure measurement device according to claim 1,
wherein the case has a spherical shape.

3. The blood pressure measurement device according to claim 2,
wherein the case has such a shape that the peripheral surface comes into plane contact with palms and fingers of the both hands in associated with a bending action of the palms and the fingers.

4. The blood pressure measurement device according to claim 3,
wherein a circumference of the peripheral surface of the case is greater than or equal to 300 mm and is less than or equal to 1000 mm.

5. The blood pressure measurement device according to claim 3,
wherein a thickness of the peripheral surface in a front-rear direction of the case is greater than or equal to 30 mm and is less than or equal to 250 mm.

6. The blood pressure measurement device according to claim 1,
wherein the case has a guide portion that informs the peripheral surface visually or tactually.

7. The blood pressure measurement device according to claim 1,
wherein the pulse wave sensor is disposed at a position corresponding to a palm of one of the hands.

8. The blood pressure measurement device according to claim 7,
wherein the pulse wave sensor is disposed at a position below a horizontal plane that bisects the case in a height direction of the case.

9. The blood pressure measurement device according to claim 7,
wherein the pulse wave sensor is disposed at a position in front of a vertical plane that bisects the case in a front-rear direction of the case.

10. The blood pressure measurement device according to claim 1,
wherein the electrocardiogram electrode includes a left hand electrode and a right hand electrode that correspond to palms or fingers of the both hands.

11. The blood pressure measurement device according to claim 10,
wherein at least a part of each of the left hand electrode and the right hand electrode is disposed above a horizontal plane that passes through the pulse wave sensor.

12. The blood pressure measurement device according to claim 10,
wherein the electrocardiogram electrode further includes an intermediate electrode for removing noise due to a body motion.

13. The blood pressure measurement device according to claim 1,
wherein the electrocardiogram electrode is embedded in the case in such a manner that a protruding height of the electrocardiogram electrode is within a range from 0.5 mm to 1.0 mm.

14. The blood pressure measurement device according to claim 9,
wherein the display portion is disposed above a horizontal plane that bisects the case in a height direction of the case and in front of the vertical plane that bisects the case in the front-rear direction of the case.

15. The blood pressure measurement device according to claim 14,
wherein the display portion is configured to display a time.

16. The blood pressure measurement device according to claim 14,
wherein the display portion is configured to inform that it is during measurement until the display portion displays a measurement result.
